(19) United States
(12) Patent Application Publication
(10) Pub. No.: US 2021/0151150 A1
(43) Pub. Date: May 20, 2021

(54) System For Interpreting And Managing Health Information

(71) Applicant: Roberta D. Powell, Cranston, RI (US)

(72) Inventor: Roberta D. Powell, Cranston, RI (US)

(21) Appl. No.: 17/248,297

(22) Filed: Jan. 19, 2021

Related U.S. Application Data
(62) Division of application No. 16/142,911, filed on Sep. 26, 2018.

(51) Int. Cl. G16H 10/60; G16H 20/10; G16H 50/30

(52) U.S. Cl. CPC G16H 10/60; G16H 50/30; G16H 20/10

(57) ABSTRACT

A patient-facing digital platform designed to promote health literacy and numeracy that helps patients access, interpret, process and contextualize personal health data so that they may manage their health conditions. The platform interprets and simplifies personal health data into simple illustrations; offers health-education videos; and translates medication information into various languages.

This application is a Divisional of Powell-20180926.

### FIELD OF THE INVENTION

The invention relates to systems and methods of clarifying health information and more particularly to clarifying, translating or simplifying medical information for laypersons. CPC schemes may include: Patient record management; Office automation, e.g. computer aided management of electronic mail or groupware; Time management, e.g. calendars, reminders, meetings or time accounting; Social work; ICT specially adapted for the handling or processing of patient-related medical or healthcare data for patient-specific data, e.g. for electronic patient records; and Computer-assisted prescription or delivery of medication, e.g. prescription filling or compliance checking.

### BACKGROUND

Most patients in the United States lack sufficient understanding of the health information around their diagnoses and conditions. Health information includes prescription dosages and instructions; lab reports; patient literature: prescribed medications; over-the-counter medications; possible interactions between drugs and with certain foods; and warnings and side effects.

According to Communicate Health (communicatehealth.com), “Only 10% of adults have the skills needed to use health information.” The remaining 90% lack the knowledge to understand and contextualize health information. The Healthcare Information and Management Systems Society states that “The ability to contextualize health information is a learned behavior; acquired through formal instruction or in medical/nursing school. Poor health numeracy and literacy skills are exacerbated by the lack of patient health education, customarily provided by registered nurses. However, due to financial constraints and a general nursing shortage, often nurses have no time to provide health education to patients. As a result, the interpretation and contextualization of health data is predominately performed by the clinicians (MD, NP, PA), who spend less than ten minutes face-to-face with their patients, leaving little time for education and dialog.” As a result, patients find themselves unable to make informed decisions about dosage, or to adhere to a prescription regimen.

Americans of various educational levels face difficulty understanding written instructions or warning labels. For example, a patient may not know that the written prescription instructions “Take 3 times per day” actually means “Take every 8 hours.” Patients may also be unable to fully understand the implications of their diagnoses or health conditions, and may consequently fail to make appropriate lifestyle and behavioral decisions. The result is worsening conditions and, in the language of hospital administrators, poor patient outcomes.

Healthcare consumers depend on clinicians or pharmacists to identify medication interactions and/or contraindications. When that fails, there is no easily accessible and reliable tool that interprets and/or clarifies medication instructions and interactions.

According to a recent study published in the journal Clinical Toxicology, “There is room for improvement in product packaging and labeling. Dosing instructions could be made clearer, especially for patients and caregivers with limited literacy or numeracy. One-third of medication errors resulted in hospital admission.” Studies have shown that patients with poor literacy have difficulty understanding medication labels.

The problem is more acute among low-literacy patients and patients for whom English is a second language. This sector struggles to interpret health data much more than those versed in healthcare or those fluent in English.

According to Univision, the Hispanic population alone accounts for over $23 billion in prescription drug sales in the United States annually, yet few, if any, pharmacy chains translate the medication labels or instructions to Spanish. The U.S. Federal government does not require pharmacies to translate prescription medication labels for non-English speakers. There is no easily accessible and reliable tool that translates, interprets and/or clarifies medication instructions and interactions for Limited-English-Speaking Patients (LEP) or those who do not speak English.

Polypharmacy is the concurrent use of multiple medications by a patient. In a 2014 report the National Institutes of Health (NIH) stated that “polypharmacy, defined as the use of multiple drugs or more than are medically necessary, is a growing concern for older adults.” Older adults with cognitive decline are particularly vulnerable to incorrect medication self-administration. According to the NIH, “Specifically, the burden of taking multiple medications has been associated with greater health care costs and an increased risk of adverse drug events (ADEs), drug-interactions, medication non-adherence, reduced functional capacity and multiple geriatric syndromes.”

Health literacy is the ability to grasp and interpret health information and data to make health decisions. Health literacy includes the elements of aural literacy, print literacy, numeracy and eHealth literacy. Aural literacy is the ability to understand what is heard. Print literacy is the ability to understand the written word or to write. Numeracy is the ability to understand numerals, calculations, logic and interpretation of numerical content. E-Health literacy refers to the ability to navigate web-based and computer-based content.

Numeracy, in general, refers to the ability to use mathematical concepts and methods. Innumeracy, in general, refers to the inability to use mathematical concepts and methods.

Health numeracy is the capacity to access, understand, process and interpret data in order to manage one's health or to make health-related decisions.

The self-management of chronic disease requires adequate health-numeracy skills. Health innumeracy may result in a patient's inability to interpret and contextualize data about their health; a difficulty making informed decisions, which can lead to a worsening of symptoms or health conditions.

In the context of this disclosure, “medication” refers to vitamin supplements, over-the-counter (OTC) medications, and prescription medications.

A “machine-readable medium storing a program for execution by processor unit of a device” is commonly referred to as an application or app. Hundreds of apps offer health information and maintenance, but each app is specialized and limited by health condition. For example, blood-pressure monitoring, glucose-level monitoring, calorie counting or exercise regimentation apps are abundant in the field, but none provide qualitative or quantitative interpretation of health values or medications nor do they warn against potential interactions.

### SUMMARY

Q2Q is a patient-facing digital platform accessible via a smartphone, tablet and computer that helps people access, interpret, process and contextualize personal health data so that they may manage their health conditions. The platform interprets and simplifies personal health data such as vital signs and lab results and converts health data into simple, color-coded illustrations and explains particular health information through animated videos. It checks for medication interactions; interprets nutrition labels as they related to chronic conditions; and translates medication information into various languages.

The platform returns information about drugs, interactions, side effects and prescription dosages, as well as information about chronic or acute health conditions. It offers relevant health education “explainer” videos about lab results and vital signs as well as evidence-based relevant health education with behavioral, lifestyle and dietary suggestions.

Q2Q integrates with electronic medical records (EMRs) through their APIs and via secure login.

Q2Q's “dashboard” window includes health numbers such as past lab values as well as current medications that may be downloaded from the patient's electronic health record.

In some embodiments the platform includes a program for receiving input in various ways, including:

Manual entry, via keypad, keyboard or similar text-entry means;

Scanned barcode entry, via camera;

Voice entry, via microphone;

Automatic download, via electronic medical record or patient portal.

The Q2Q platform is multilingual; it accepts and delivers information in multiple languages, via text or voice input. It also translates information into various languages using the Google Translate API. In some embodiments, the language used for information entry is specified by the user; in other embodiments the language is recognized by the program in the app. One skilled in the art understands that information typed, scanned, spoken, or downloaded may be interpreted by a program to determine the language of the information. Once the language of the information is determined the output data may be provided in the same language or may be translated to another language.

Q2Q uses artificial intelligence (AI) to analyze entered data, such as patient history, to interpret and extract values. Entered data is captured in a database. It uses character and voice recognition to extract relevant values from photographs of patient lab reports and verbal inquiries; analyzes extracted data; and presents information in user-friendly graphical elements.

One skilled in the art understands the ability of AI to recognize spoken words, scanned images of text and convert the information into machine-readable medium.

In another iteration of the embodiment data entered into the app by the aforementioned methods is analyzed and converted into a simplified visual display including graphics and text, providing analysis of a patient's medical history.

Other objects and features will become apparent from the following detailed description considered in conjunction with the accompanying drawings. Drawings are intended to illustrate rather than define the limits of the invention.

### BRIEF DESCRIPTION OF THE DRAWINGS

To assist those of skill in the art in making and using the disclosed invention and associated methods, FIGS. 1-5 show the user interfaces of an example embodiment of the present disclosure, as shown displayed on a provided smartphone.

FIG. 1 is a plan view of a user interface screen as shown displayed on a provided smartphone.

FIG. 2 is a plan view of three related user interface screens.

FIG. 3 is a plan view of three related user interface screens.

FIG. 4 is a detail view and a plan view of three related user interface screens with a graphic display interpreting results of entered data.

FIG. 5 is a plan view of three related user interface screens showing interpreted results of entered data.

FIGS. 6-13 show the user interfaces of a second example embodiment of the present disclosure, as shown displayed on a provided smartphone.

FIG. 6 is a plan view of a user interface screen of a second embodiment of the disclosure.

FIG. 7 is a plan view of three related user interface screens of the embodiment of FIG. 6.

FIG. 8 is a plan view showing results of user-entered information of FIG. 6.

FIG. 9 is a plan view of a user interface screen of the embodiment of FIG. 6 in which an example of a search result appears.

FIG. 10 is a plan view showing translation options of a user-interface screen of the embodiment of FIG. 6.

FIG. 11 is a plan view of a text-magnify option of the user-interface screen of the embodiment of FIG. 6.

FIG. 12 is a plan view of an interpretation feature of the user-interface screens of the embodiment of FIG. 6.

FIG. 13 is a plan view of an interaction checker of the user-interface screen of the embodiment of FIG. 6.

FIGS. 14-16 are flowchart views of user interaction with an iteration of the embodiment.

FIG. 14 is a flowchart of user interaction with the embodiment.

FIG. 15 is a flowchart of user interaction with the embodiment.

FIG. 16 is a flowchart of user interaction with the embodiment.

FIGS. 17-20 are flowchart views of user interaction with an iteration of the embodiment.

FIG. 17 is a flowchart of user interaction with an iteration of the embodiment.

FIG. 18 is a flowchart of user interaction with an iteration of the embodiment.

FIG. 19 is a flowchart of user interaction with an iteration of the embodiment.

FIG. 20 is a flowchart of user interaction with an iteration of the embodiment.

### DESCRIPTION

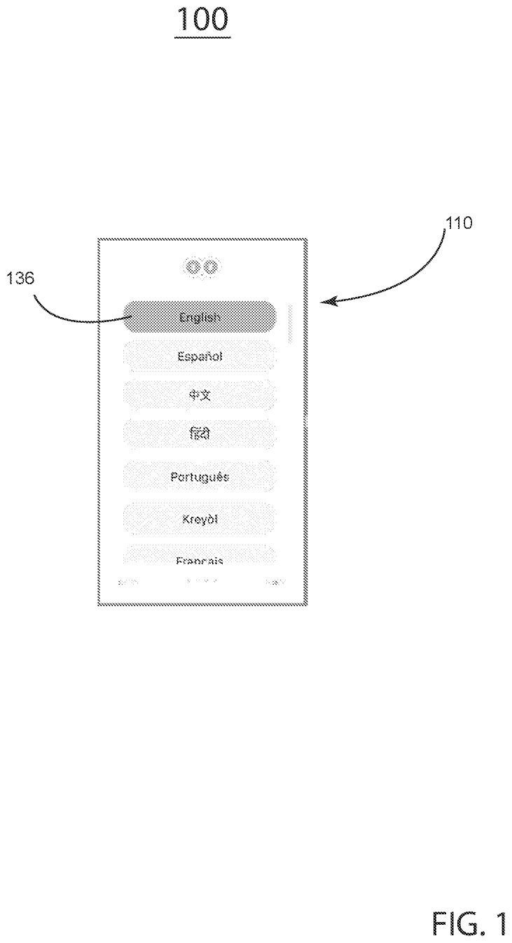

In an embodiment 100, FIG. 1 shows the app's initial screen 110 for choosing a primary language, in this case English 136.

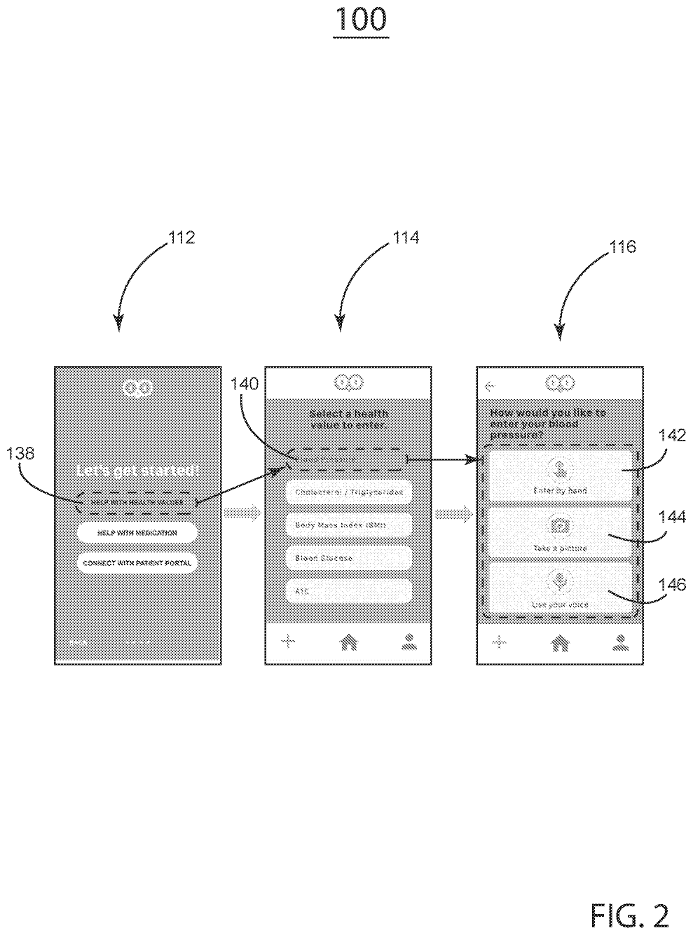

FIG. 2 shows the start 112 of a program. A program-feature choice 138 has been selected. A specific health value 140 is selected from the health-value selector 114, giving the further option of selecting a mode selector for entering data 116. Options for entering data are manual entry 142; scanned entry 144; and spoken entry 146.

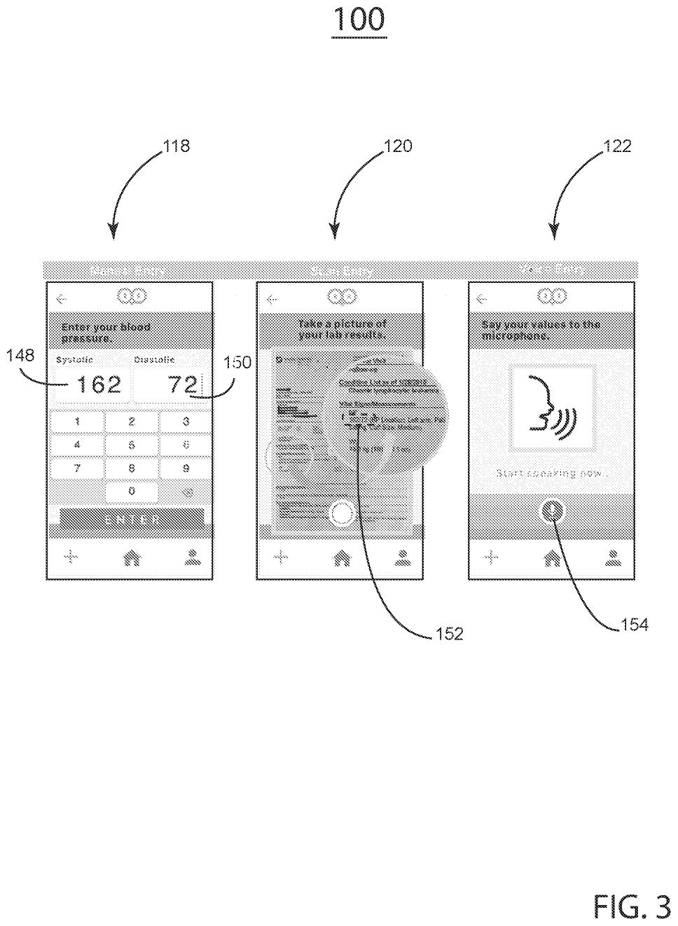

FIG. 3 shows the app's manual-entry option 118 and a specific manual-entry example 148, 150. A scan-entry option 120 is shown on another app screen. In this case, for example, the user has scanned their lab results 152. A voice-entry option 122 is shown in a third app screen. In this case the user has spoken an entry 154.

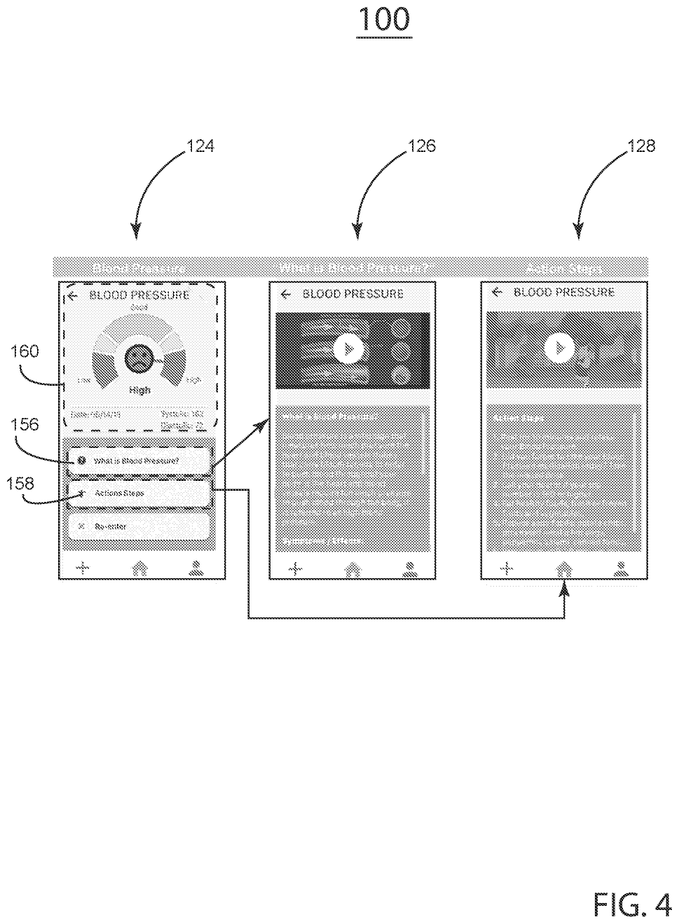

FIG. 4 shows a graphic display 124 interpreting results of entered data. A graphic design shows a high blood pressure 160 and a button for more information 156 and another button 158 with suggested action steps. Selecting a button for more information 156 brings up information about the chosen topic 126. Selecting the “Action Steps” button 158 returns suggested action steps 128.

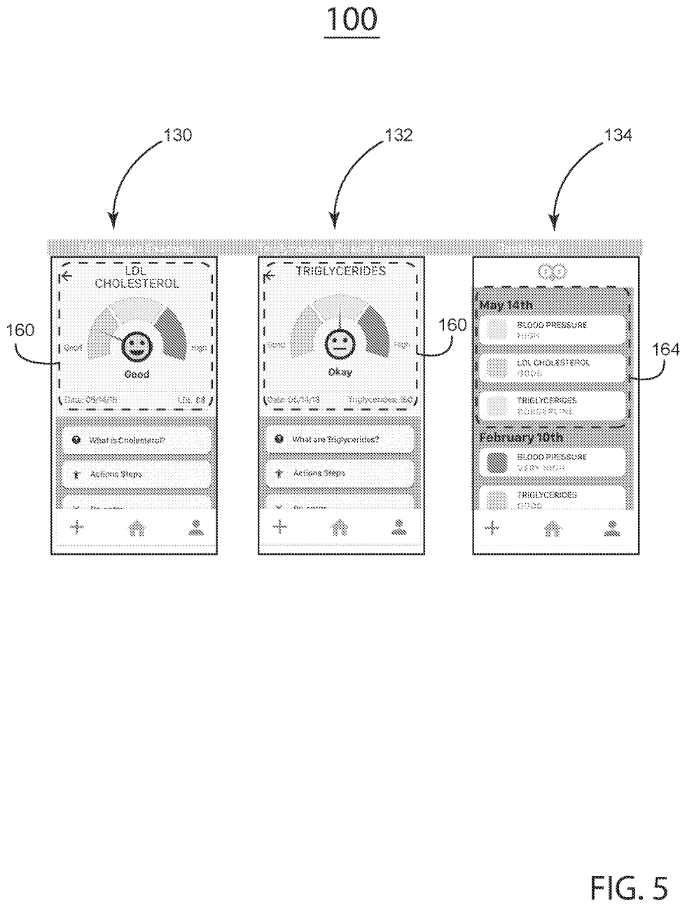

FIG. 5 shows a graphic display 130 interpreting results of entered data, in which a graphic design interprets results of entered data for, example, LDL cholesterol 160 and triglycerides 132. The third illustration shows a navigation screen 134 for viewing historical data 164.

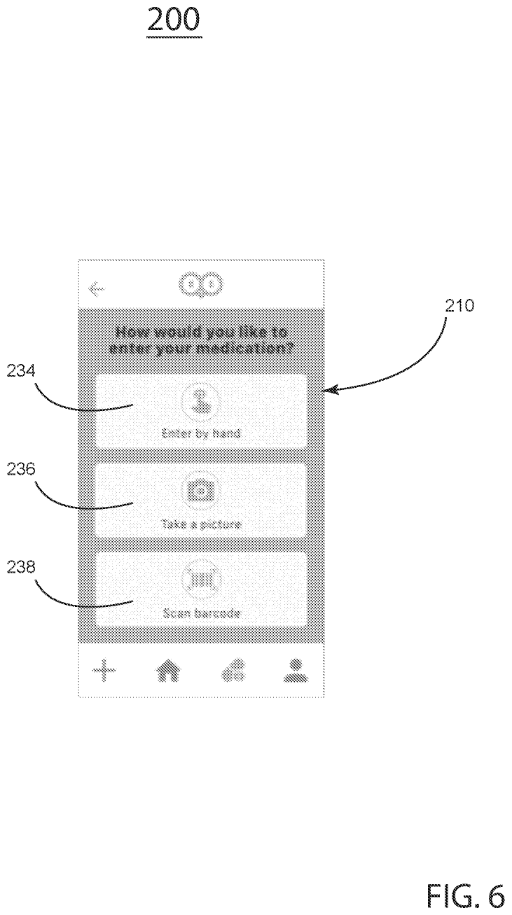

In a second iteration 200, FIG. 6, a user may select from various medication-entry methods 210 including manual medication entry 234, photograph entry 236 or barcode-scan entry 238.

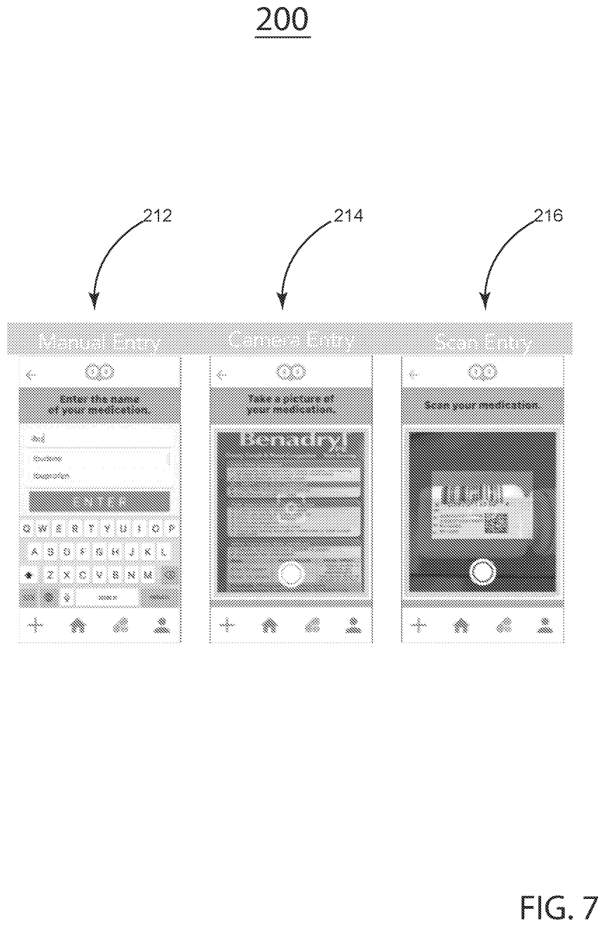

FIG. 7 200 shows screens that are the result of each choice. In the manual medication entry screen 212 the user types a medication using an on-screen keyboard. In the camera-entry screen 214 the user has taken a picture of a medication label. In the scanned barcode entry screen 216 the user scans a medication via their (provided) smartphone's camera.

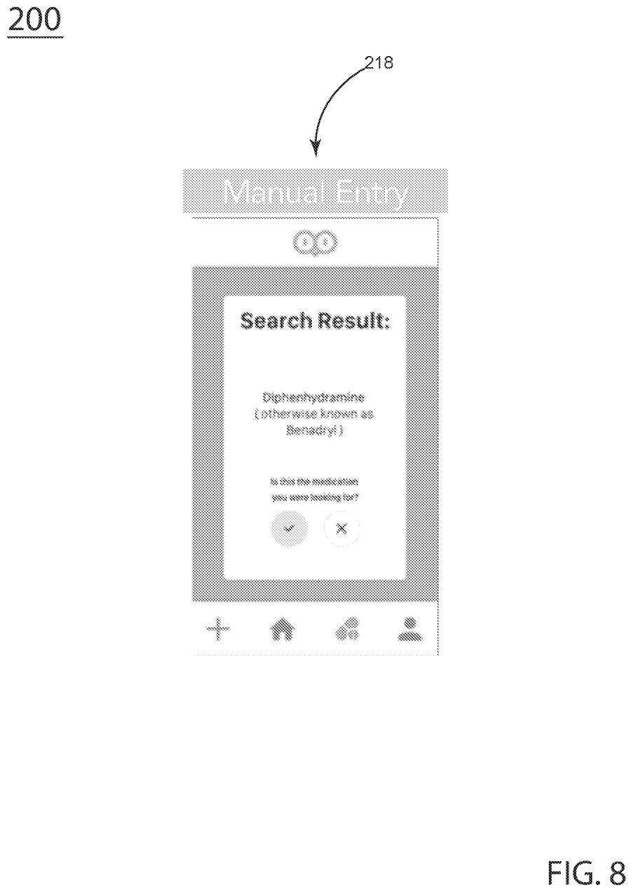

FIG. 8 200 shows a manual-entry result screen 218.

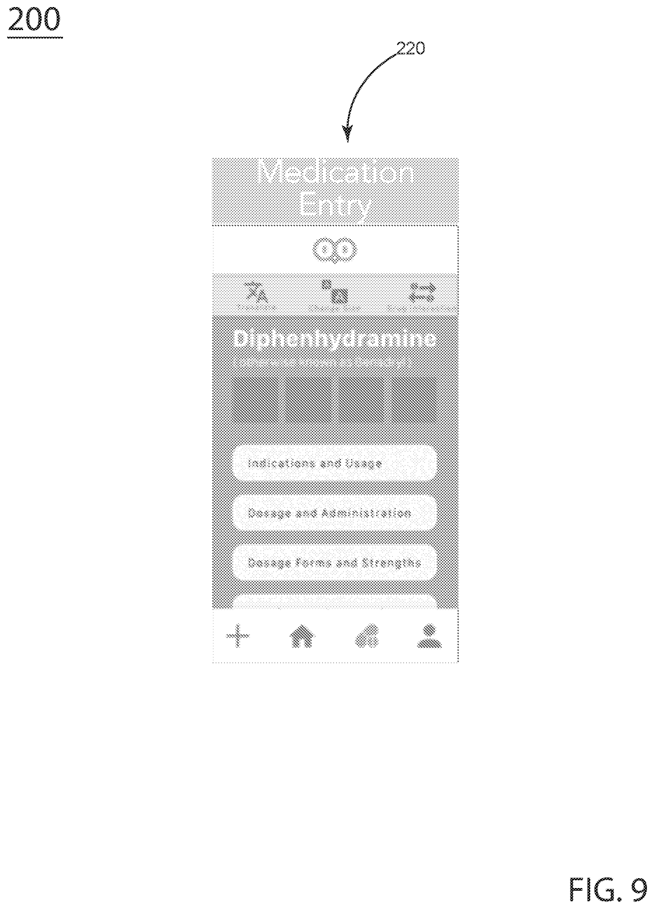

FIG. 9 200 shows an example of a search result 220.

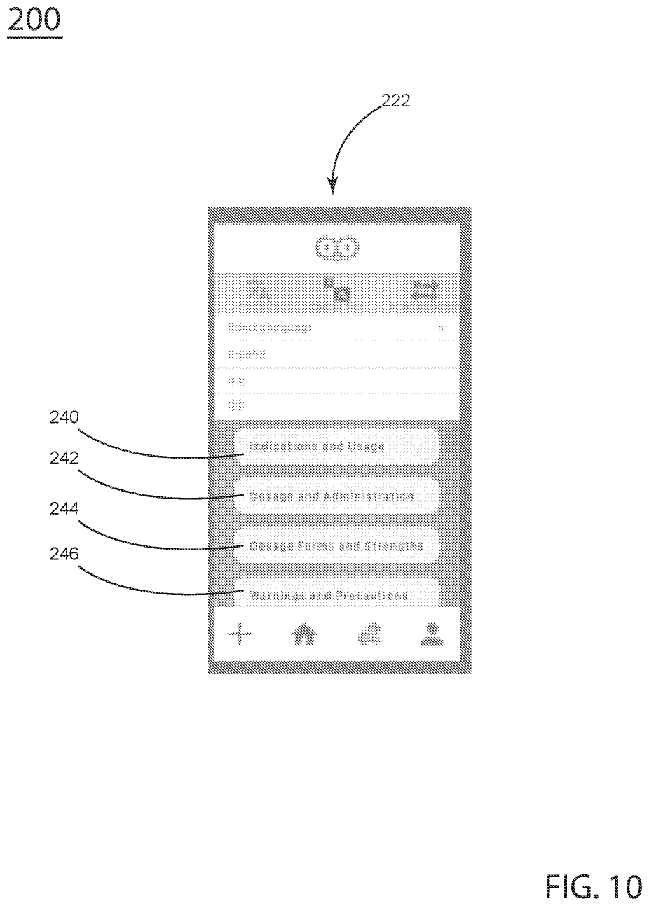

FIG. 10 200 shows the translation screen 222, where one may choose to translate indications and usage 240; dosage and administration 242; dosage forms and strengths 244; or warnings and precautions 246.

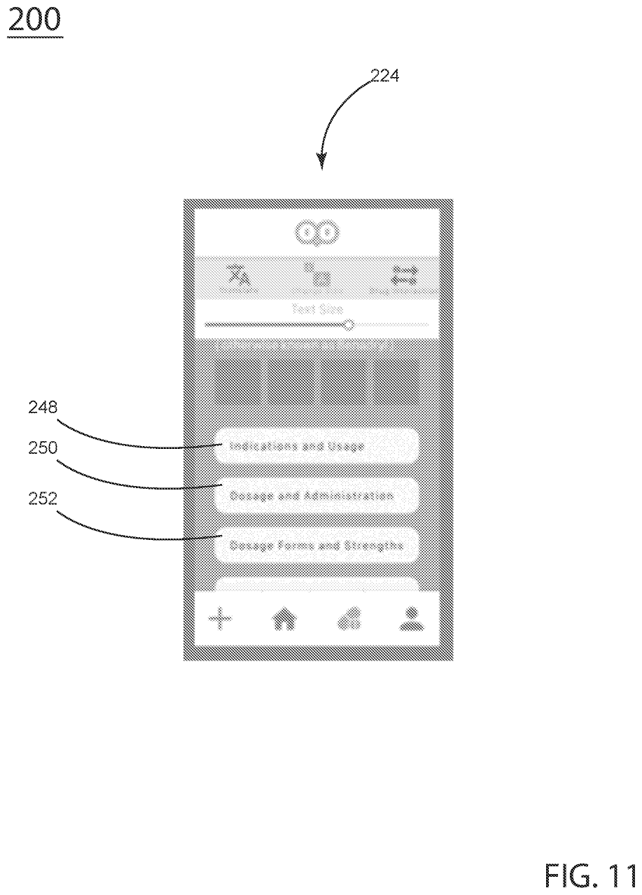

FIG. 11 200 shows the text-magnify option 224 in which options 248, 250, 252 are shown magnified.

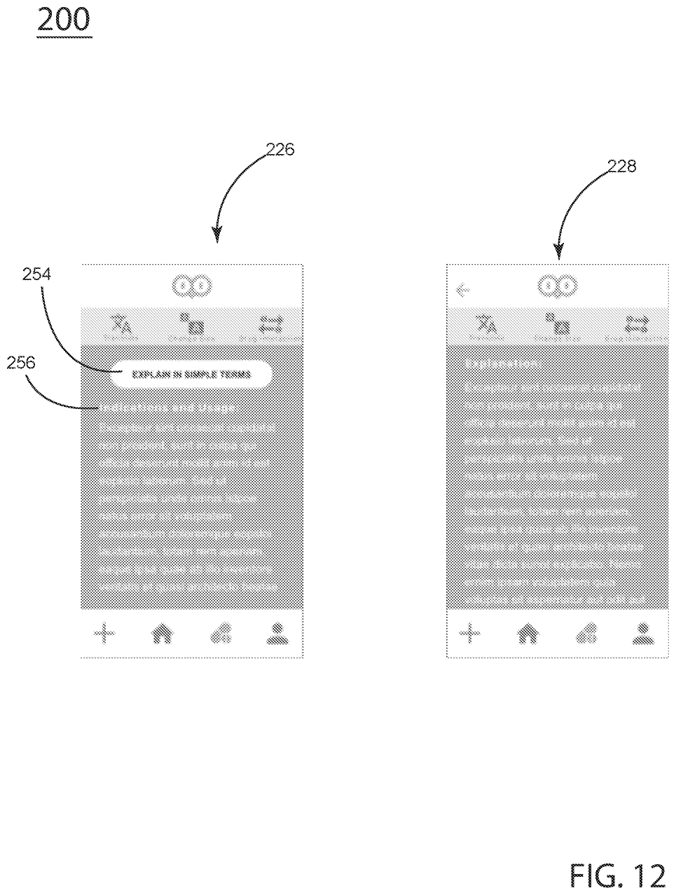

FIG. 12 200 shows the option to explain entered information in simple terms 226. The information that was entered 256 may be simplified by tapping an explanation button 254. Once that button is tapped, the entered information is re-interpreted in simplified terms 228.

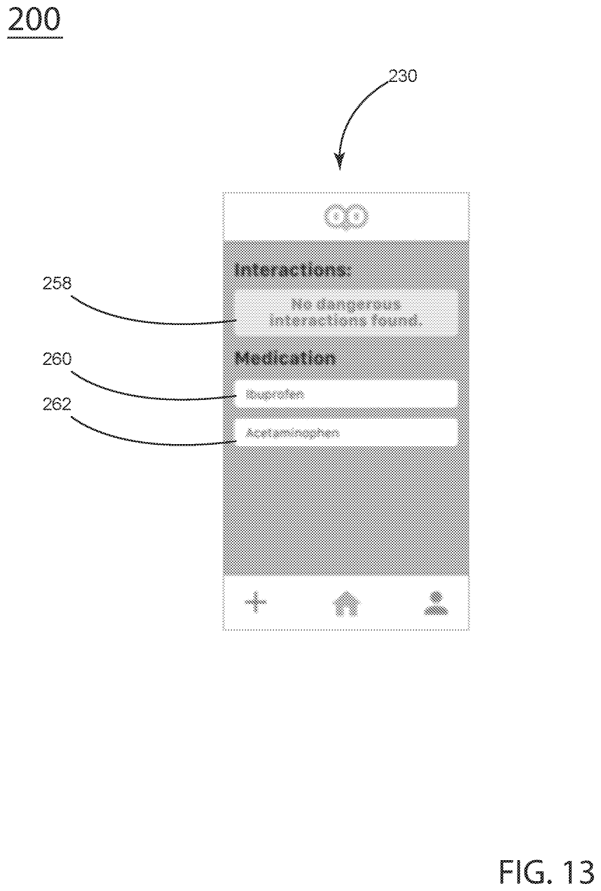

FIG. 13 200 shows an interaction checker 230 with example medications 260, 262 entered and interactions 258 determined.

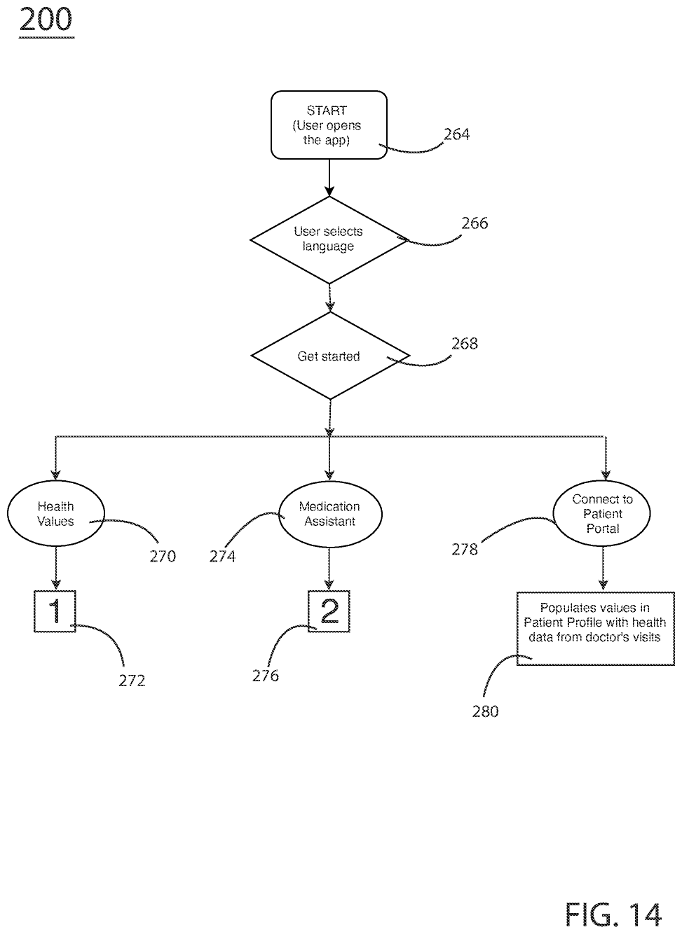

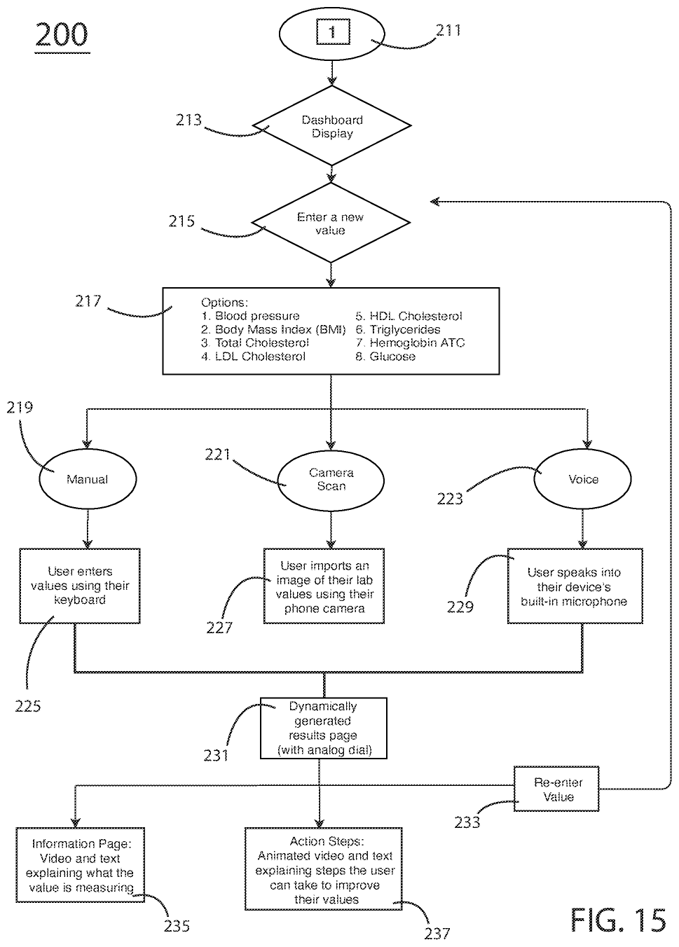

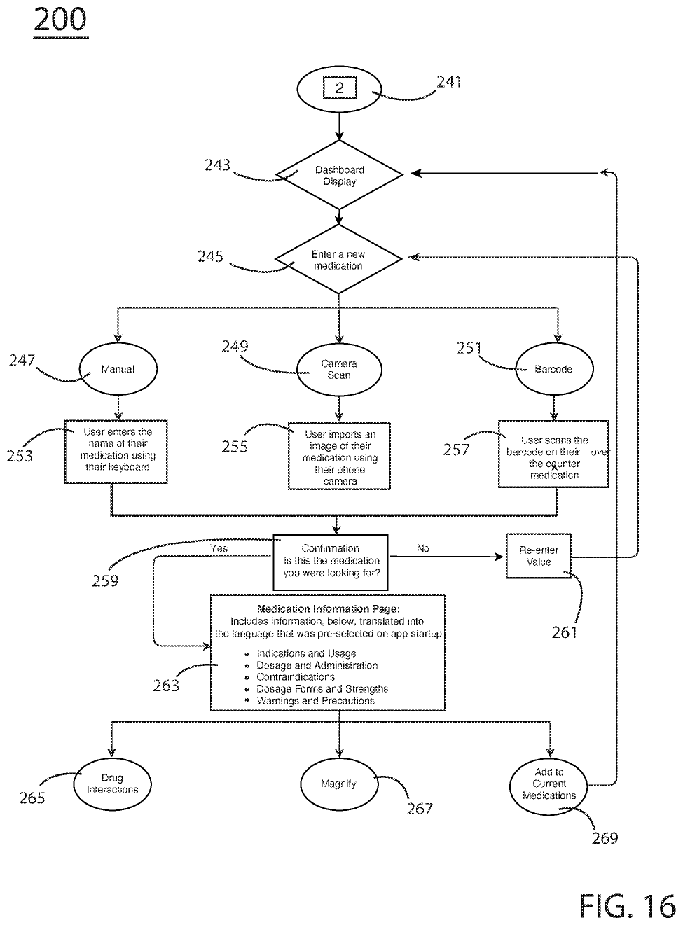

In FIG. 14 200, a flowchart illustrates the progression of steps from a user's perspective. Upon opening the app 264 on their device, a user selects a language 266. From there they choose from three branches 268 to obtain medical information. Branch 1, “Health Values” 270, leads to an input screen (FIG. 15) for entering values such as blood pressure, cholesterol, etc.; Branch 2 (FIG. 14) “Medication Assistant” 274, leads to an input screen (FIG. 16) for entering medications; and a third branch (FIG. 14) 278 leads to a patient-portal connection which connects to a patient's EHR portal, where users enter their credentials to access their medical record 280. (Branch 3 is not illustrated further).

The flowchart in FIG. 15 200 illustrates results of choosing the first branch, “Health Values” numeral 1, 211. The dashboard 213 loads, showing the user's previous entries. In some embodiments, the app checks for connection to a patient portal and if found, allows the patient to log in to retrieve electronic health record information such as recent visits. The user enters a value to be interpreted 215, for example blood pressure, cholesterol, or other data 217. Options for input include manual input 219, in which the user types a value 225; camera-scan 221, in which the user employs the (provided) camera app on their smartphone to photograph or import 227 a photograph of, for example, lab values; and voice entry 223, wherein the user speaks information 229 into their smartphone using the smartphone's provided voice app. Once the health data is entered, the app generates information about each entry 231, interpreting results via a graphic design such as a dial 231, or as text; or in the form of an educational video 235 or a video of action steps 237. Subsequent options include re-entering a corrected value 233 to start the process again.

The flowchart in FIG. 16 200 shows events after the user chooses the “Medication Assistant” branch 2, 241. The dashboard 243 loads, showing the user's current medications. In some embodiments, the app checks for connection to a patient portal and if found, allows the patient to log in to retrieve electronic health record information such as recent visits. The user enters a medication to be interpreted 245. Options for input include manual input 247, in which the user types 253 the name of the medication; camera-scan 249, in which the user employs the (provided) camera app on their smartphone to photograph or import 255 a photograph of a medication label; and barcode-scan 251, in which a user scans the barcode 257 on their over-the-counter medication using the app's barcode-scanning feature. into their smartphone using the smartphone's provided voice app. Once a medication is entered, the app seeks confirmation 259. If incorrect, the app re-routes 261 to the medication-entry step 245. If the entered medication is confirmed by the user as correct, the app generates information about that medication 263 including indications; values and types of dosage; administration; contraindications; and precautions and warnings. If the entered medication has contraindications or possible interactions with their current medications, a pop-up box 265 will appear with this information. Users may adjust the text size 267 of the generated results by using a graphical slider. They may add 269 this medication to list of current medications.

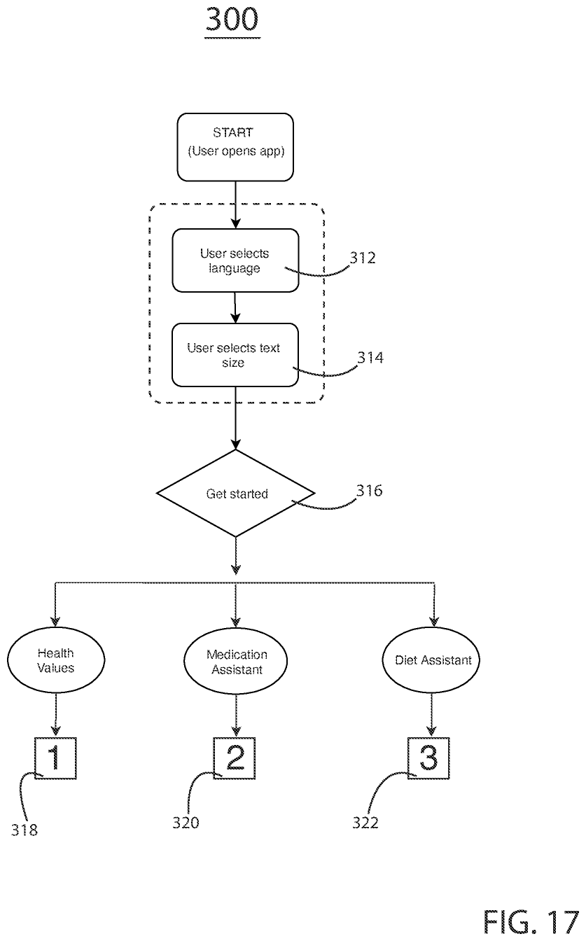

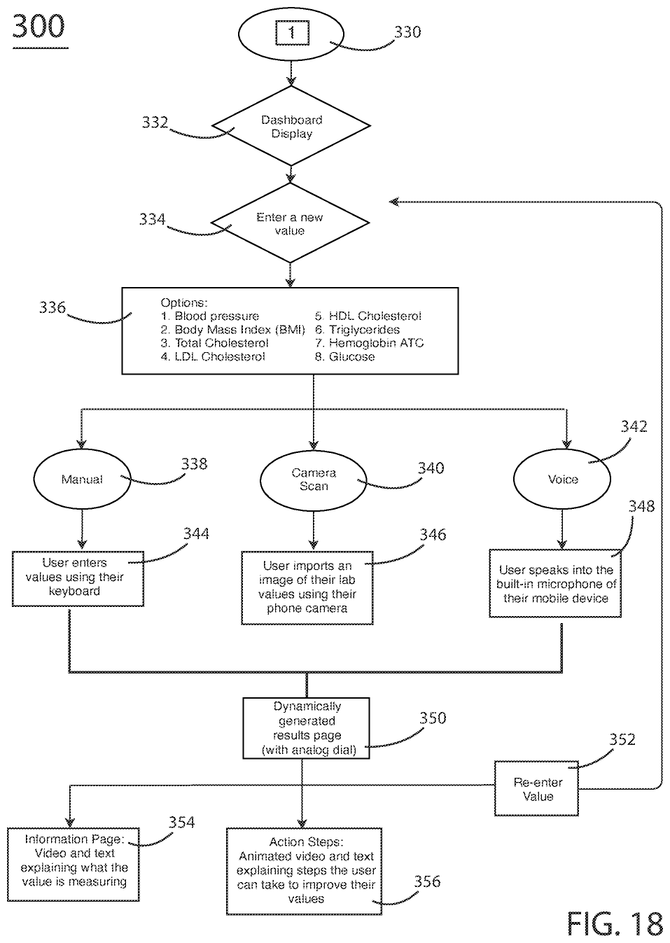

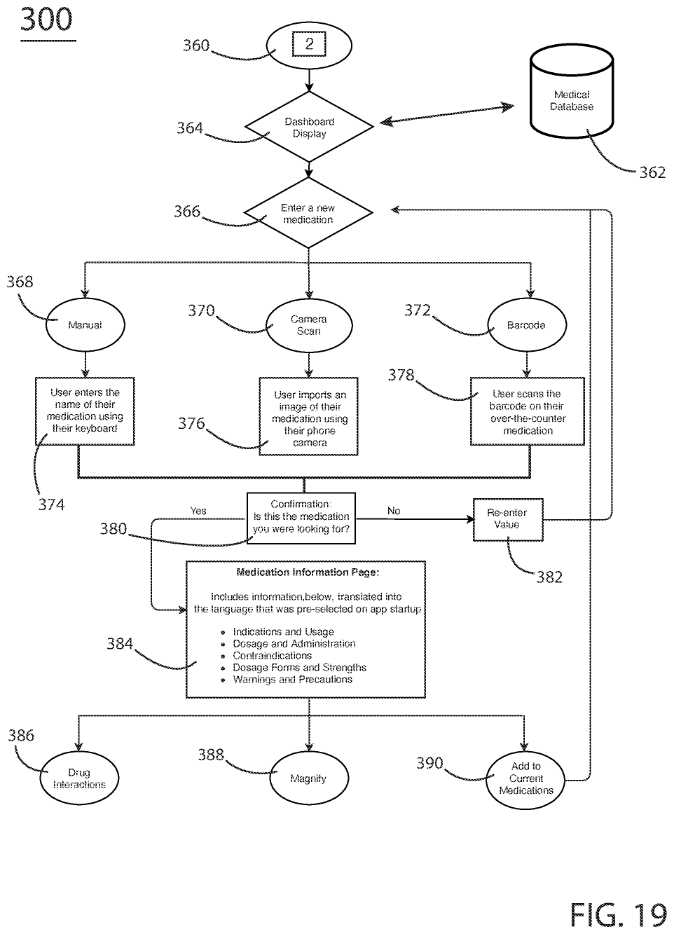

FIG. 17 illustrates a third example iteration, 300. Upon opening the app on their device a user selects a language 312 and a text size 314, bringing them to a “Get Started” window 316. From there the user may choose from three branches to obtain medical information. Branch 1, “Health Values” 318, leads to an input screen (FIG. 18) for entering values such as blood pressure, cholesterol, etc. Branch 2, “Medication Assistant,” FIG. 17, 320 leads to an input screen that starts a process (FIG. 19) for obtaining medication information. Branch 3, “Diet Assistant” FIG. 17, 322 leads to an input screen that starts a process by which the user can check drug interactions.

In FIG. 18, 300 a “Health Values” branch 1, 330 illustrates the interpretation of user-entered health data. The dashboard 332 loads, showing the user's previous entries. In some embodiments, the app checks for connection to a patient portal and if found, allows the patient to log in to retrieve electronic health record information such as recent visits. The user enters a value to be interpreted 334, for example blood pressure, cholesterol, or other data 336. Options for input include manual input 338, in which the user types a value 344; camera-scan 340, in which the user employs the (provided) camera app on their smartphone to photograph or import 346 a photograph of, for example, lab values; and voice entry 342, wherein the user speaks information 348 into their smartphone using the smartphone's provided voice app. Once the health data is entered, the app generates information about each entry, interpreting results via a graphic design such as a dial 350, or as text; or in the form of an educational video 354 or a video of action steps 356. Subsequent options include re-entering a corrected value 352 to restart the process.

FIG. 19, 300 shows events after the user chooses the “Medication Assistant” branch 2, 360. The dashboard 364 loads, showing the user's current medications. In some embodiments, the app checks for connection to a patient portal and if found, allows the patient to log in to retrieve electronic health record information such as recent visits. The user enters a medication to be interpreted 366. Options for input include manual input 368, in which the user types 374 the name of the medication; camera-scan 370, in which the user employs the (provided) camera app on their smartphone to photograph or import 376 a photograph of a medication label; and barcode-scan 372, in which a user scans the barcode 378 on their over-the-counter medication using the app's barcode-scanning feature. Once a medication is entered, the app seeks confirmation 380. If incorrect, the app re-routes 382 to the medication-entry step 366. If the entered medication is confirmed by the user as correct 380, the app generates information about that medication 384 including indications; values and types of dosage; administration; contraindications; and precautions and warnings. If the entered medication has contraindications or possible interactions with their current medications, a pop-up box 386 will appear with this information. Users may adjust the text size 388 of the generated results by using a graphical slider. They may add 390 this medication to list of current medications.

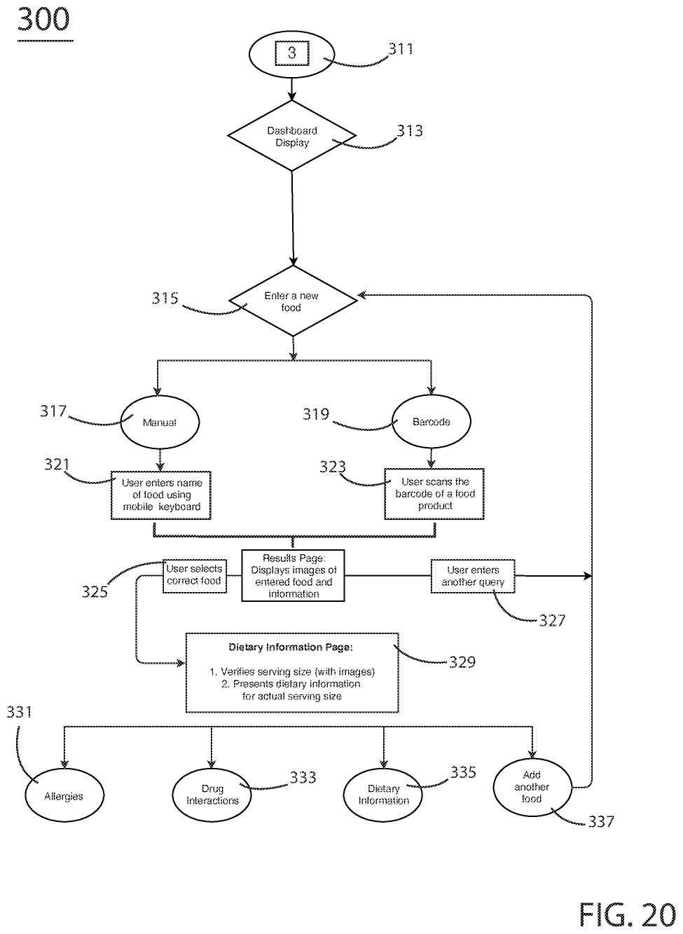

FIG. 20, 300 illustrates events after the user chooses the “Diet Assistant” branch 3, 311. The dashboard 313 loads, showing the user's caloric intake for a defined duration, as well as relevant data on fat, cholesterol, sodium and other intake. The user enters a food 315 in one of two ways: manual input 317, in which the user types 321 the name of the food; or barcode-scan 319, in which the user scans the barcode 323 of their food product using the app's barcode-scanning feature. Once a food is entered, the app presents an image of the entered food 325 for confirmation. If incorrect, the app re-routes 327 to the food-entry step 315. If the entered food is confirmed by the user as correct 325, the user chooses the generated image and a dietary information page 329 opens, which verifies serving size and other dietary information. An option appears 335 to add dietary information to a daily sum for values relevant to medical history. If the entered food contains allergens or is commonly processed with known allergens, the app generates an allergy warning 331. If the entered food contains ingredients that may interact with the user's current medications a drug interaction warning 333 appears. Concurrent with these options is an option to add a new food 337 to begin the Diet Assistant process on that entry.

In all the above iterations (100-300), a menu icon appears at all times at a corner of each screen, allowing any of these options at any point: “Home” to return to the Get Started page; “Profile” to return to the dashboard; “Add Medication” to return to the Medication Assistant; or “Back” to return to the previously visited page.

These embodiments are understood to be exemplary and not limiting. Additions and modifications to what is expressly described here are understood to be included within the scope of the invention. The features of the various embodiments described here are not mutually exclusive and can exist in various combinations and permutations, even if such combinations or permutations are not made express here, without departing from the spirit and scope of the invention.

1. A system for interpreting and managing health information comprising:
a user interface for choosing an operative language; and
a user interface for manual data entry of numeric vital signs; and
information derived from manual data entry is converted to non-transitory computer-readable medium storing instructions; and
said instructions convert said numeric vital signs into at least one color-coded analog graphic representation; and
said instructions look up, locate and display an explanation of said analog graphic representation in text format; and
said instructions look up, locate and display action steps, in text format, that correspond with said vital signs; wherein
the data entered by manual data entry is interpreted and displayed as an analog graphic representation in combination with explanation of the graphic in text format and action steps in text format in the operative language.

2. The system for interpreting and managing health information of claim 1 further comprising:
a user interface for scanned image data entry.

3. The system for interpreting and managing health information of claim 1 further comprising:
a user interface for barcode scanned data entry.

4. The system for interpreting and managing health information of claim 1 further comprising:
a user interface for manual data entry of medical laboratory results.

5. The system for interpreting and managing health information of claim 1 further comprising:
a user interface for scanned image data entry of medical laboratory results.

6. The system for interpreting and managing health information of claim 1 further comprising:
a user interface for scanned-barcode data entry of medical laboratory results.

7. The system for interpreting and managing health information of claim 1 further comprising:
vital signs converted to whole numbers for display in said analog graphic representation.

8. A non-transitory computer-readable medium storing instructions that when executed by a computer cause the computer to perform operations of a method comprising:
providing the selection of health values; and
selecting vital sign to be evaluated; and
selecting manual data entry; and
selecting camera-scanned data entry; and
selecting vocal data entry; and
generating a color-coded analog graphic representation from the entered data; and
displaying text of an explanation of the color-coded analog graphic in the operative language; and
displaying action steps in text format in the operative language; wherein
a health value is denoted by a chosen vital sign and mode of data entry that is converted to a graphic analog representation with textual explanations of the vital sign data and action steps recommended.
